(19) United States
(12) Patent Application Publication
(10) Pub. No.: US 2006/0015051 A1
(43) Pub. Date: Jan. 19, 2006

(54) Gold tattoo

(76) Inventor: Sarah Davanzo, New York, NY (US)

Correspondence Address: BIRCH STEWART KOLASCH & BIRCH, PO BOX 747, FALLS CHURCH, VA, 22040-0747, US

(21) Appl. No.: 10/644,031

(22) Filed: Aug. 20, 2003

(30) Foreign Application Priority Data
Aug. 20, 2002 (ZA) 2002/6650

(51) Int. Cl. A61F 13/00

(52) U.S. Cl. 602/41

(57) ABSTRACT

An article of adornment in the form of a temporary gold tattoo comprises a gold leaf applied to a synthetic medical skin using Japan Gold Size, and die-cut to the desired shape. The synthetic medical skin is sterile, hypoallergenic and allows skin to which the adornment is adhered to breathe. Thus, the resulting temporary gold tattoo is thin and comfortable to wear and yet stays firmly in place, even withstanding normal bathing for a few days.

### BACKGROUND OF THE INVENTION

THIS invention relates to an article of adornment, particularly to a temporary gold tattoo.

In recent years various attempts have been made to manufacture temporary “gold tattoos” or similar products primarily as make-up and adornment for fashion photo-shoots, fashions shows, theatrical performances and adornment. The objective has been to invent a tattoo or decal which:
Is made of real gold (ideally 22-24 ct).
Is gold metallic in colour (not just gold coloured).
Can be easily and quickly applied.
Is applied in a uniform manner in shapes that can be repeated (i.e. not painted individually).
Comes in a variety of desirable shapes that can be combined to create individual motifs.
Flexible and moves with the skin.
Is safe to wear (sterile, hypoallergenic and allows the skin to breath).
Is thin and comfortable to wear.
Stays firmly in place (does not slide around).
Can easily be removed without irritating the skin, leaving no residue.
Can withstand normal bathing.
Lasts for a few days.

To achieve such an adornment, various methods have been used in the past, each with their individual drawbacks:
Gold Body Paint—Painting the body with gold coloured paint or theatrical make-up is the most common method of achieving the look of a “gold tattoo” or gold coloured body adornment. However, gold coloured paint is not made of real gold, as real gold cannot be made into a paint which can be placed on the skin without irritation. In addition, painting the body with gold coloured paint is very laborious and time consuming. The motif is applied by hand, and the paint does not last long, washing off easily. In fact, it is dangerous to have large parts of the body painted with gold paint because the skin does not breathe.
Gold leafed decals—Plastic and paper decals have been gold leafed, or painted gold, and applied to the body either with decal adhesive or eyelash glue. Sometimes a “spray mount” adhesive is used, but this can irritate the skin and leaves a sticky residue once removed. The concern with this process is that the adhesive on decals is not safe for application to the skin, especially for a long duration. In addition, paper decals cannot withstand bathing and thus do not last. Eyelash glue leaves a residue.
Gold leaf applied directly to the skin with various glues—Motifs can be drawn onto the body with eyelash or surgical glue, and the gold leaf can be applied afterwards. The drawback with this method is that the motif, again, is applied by hand, laboriously. The gold leaf application is uneven (e.g. it looks like gold flakes, not a smooth gold finish) and is very wasteful, thus the motifs are limited.
Gold coloured decal tattoos—“Tattoo decals” which can be applied with water to the surface of the skin and which can be washed off, can be found in gold colours. These are manufactured in an array of shapes and sizes. However, the gold is merely gold ink, not real gold, and the colour is a brownish-gold, not metallic.

Therefore, the present invention seeks to provide an improved article of adornment in the form of a temporary gold tattoo.

### SUMMARY OF THE INVENTION

According to the present invention there is provided an article of adornment comprising:
a carrier layer having a first adhesive on one side thereof, wherein the carrier layer is sterile, hypoallergenic and allows skin to which the adornment is adhered to breathe;
a removable covering layer which covers the first adhesive when the adornment is not in use and which can be removed to expose the first adhesive when the adornment is to be used; and
a metal layer connected to the carrier layer.

The metal layer may include gold, silver, platinum or copper.

The metal layer is preferably a 22 ct or 24 ct gold leaf.

The metal layer may be connected to the carrier layer using a second adhesive, such as Japan Gold Size.

The carrier layer may be waterproof.

The carrier layer is preferably a synthetic medical skin.

Preferably, the carrier layer and metal layer are substantially the same size as they have been cut after they have been connected to one another.

According to the present invention there is further provided a method of making an article of adornment, the method comprising the steps of:
applying a metal layer to a carrier layer, the carrier layer having a first adhesive on one side thereof, wherein the carrier layer is sterile, hypoallergenic and allows skin to which the adornment is adhered to breathe; and
cutting the combined metal layer and carrier layer into a desired shape.

The metal layer may be connected to the carrier layer using an adhesive, typically Japan Gold Size.

Preferably, the combined metal layer and carrier layer is die-cut.

### BRIEF DESCRIPTION OF THE DRAWINGS

FIG. 1 illustrates a carrier layer and gold leaf layer used to form the article of adornment of the present invention before the layers have been connected together;

FIG. 2 illustrates the layers of FIG. 1 connected together with the shape of the tattoo cut into the layers; and

FIG. 3 illustrates the temporary gold tattoo of the present invention with the removable covering layer in the process of being removed.

### DESCRIPTION OF AN EMBODIMENT

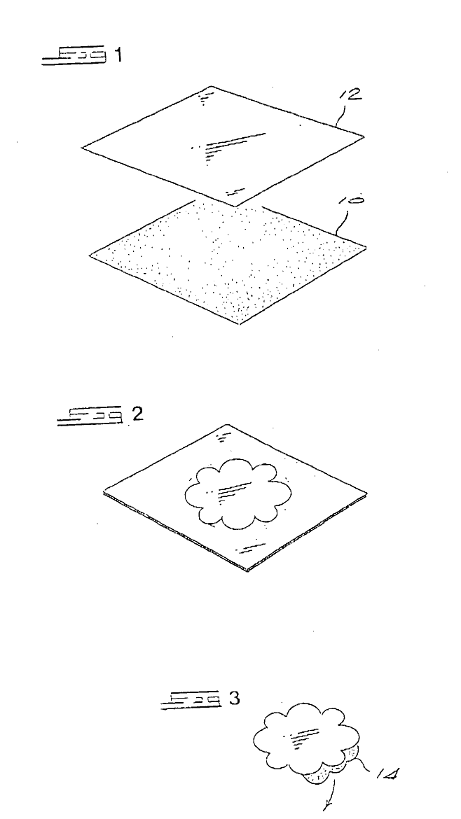

Referring to FIG. 1, the article of adornment of the present invention, in the form of a temporary gold tattoo, is formed from a carrier layer 10 and a metal layer 12.

The carrier layer 10 is a synthetic medical skin which is sterile, hypoallergenic and which allows the skin to breathe when it is applied to the skin.

Examples of these synthetic medical skins are Tegaderm™ sold by 3M™ and Opsite™ sold by Smith & Nephew™.

The carrier layer 10 includes a first adhesive on one side thereof, which adhesive is used to adhere the carrier layer to the skin of a user. While the carrier layer is not in use, a removable covering layer (not shown) covers the adhesive.

The metal layer 12 is preferably a gold leaf layer, but could be another type of metal layer used for adornment, such as a silver, platinum, copper, for example.

When forming a temporary gold tattoo, the metal layer includes at least some gold content, but is preferably 22 ct or 24 ct gold.

In order to connect the gold layer to the carrier layer, a second adhesive is used. In the prototype of the present invention, the second adhesive is Japan Gold Size.

Japan Gold Size falls into the category of cooked oil and resin varnishes and is composed of Tung oil, Phenolic resin, driers and white spirit (refined “mineral turps”). Tung oil is oil of vegetable origin derived from nuts. Phenolic resin is a modern synthetic resin related to benzene. Driers are derived from metallic salts such as cerium and/or zirconium.

The Japan Gold Size is spread over the surface of the synthetic medical skin 10 and once it is dry the gold leaf 12 is applied to the surface of the dry Japan Gold Size.

Referring to FIG. 2, the gold surface is covered with a non-stick paper and the combination is placed in a die-cut that cuts out the required shape of the temporary gold tattoo.

Once the shape has been cut out, the resulting gold tattoo is ready to be used.

In order to be applied, a user simply cleans and dries the area where the temporary gold tattoo is to be applied. The removable covering layer 14 is peeled away to expose the adhesive.

The temporary gold tattoo is applied onto the skin and is smoothed from the centre towards the edges.

Because these synthetic medical skins are waterproof, it has been found that the temporary gold tattoo will last between 5 and 7 days with normal bathing as long as the surface of the temporary gold tattoo is not scrubbed whilst bathing.

It will be appreciated that slightly alternative methods of making the temporary gold tattoo could be implemented. For example, the gold leaf may be die-cut before being applied to the carrier layer. This would not detract from the essence of the invention.

It will also be appreciated that the temporary gold tattoo is made of real gold and is not just gold coloured.

The tattoo can be easily and quickly applied in a uniform manner and in shapes that can be repeated. Because of the ease of application, a variety of desirable shapes can be combined to create individual motifs.

Most importantly, the temporary gold tattoo is safe to wear as it is sterile, hypoallergenic and allows the skin to breathe.

In addition, the temporary gold tattoo is thin and comfortable to wear and yet stays firmly in place, even withstanding normal bathing for a few days.

Finally, the temporary gold tattoo can easily be removed leaving no residue.

I claim:

1. An article of adornment comprising:
a carrier layer having a first adhesive on one side thereof, wherein the carrier layer is sterile, hypoallergenic and allows skin to which the adornment is adhered to breathe;
a removable covering layer which covers the first adhesive when the adornment is not in use and which can be removed to expose the first adhesive when the adornment is to be used; and
a metal layer connected to the carrier layer.

2. An article of adornment according to claim 1, wherein the metal layer includes gold, silver, platinum or copper.

3. An article of adornment according to claim 2, wherein the metal layer is a 22 ct or 24 ct gold leaf.

4. An article of adornment according to claim 1 wherein the metal layer is connected to the carrier layer using a second adhesive.

5. An article of adornment according to claim 4, wherein the second adhesive is Japan Gold Size.

6. An article of adornment according to claim 1 wherein the carrier layer is waterproof.

7. An article of adornment according to claim 1 wherein the carrier layer is a synthetic medical skin.

8. An article of adornment according to claim 1 wherein the carrier layer and metal layer are substantially the same size as they have been cut after they have been connected to one another.

9. A method of making an article of adornment, the method comprising the steps of:
applying a metal layer to a carrier layer, the carrier layer having a first adhesive on one side thereof, wherein the carrier layer is sterile, hypoallergenic and allows skin to which the adornment is adhered to breathe; and
cutting the combined metal layer and carrier layer into a desired shape.

10. A method according to claim 9 wherein the metal layer is connected to the carrier layer using an adhesive.

11. A method according to claim 10 wherein the adhesive is Japan Gold Size.

12. A method according to claim 10 wherein the combined metal layer and carrier layer is die-cut.
